(19) United States
(12) Patent Application Publication
(10) Pub. No.: US 2012/0121745 A1
(43) Pub. Date: May 17, 2012

(54) AGENTS FOR THE CONTROL OF LIMNOPERNA SP.

(75) Inventors: Sarahann Rackl, Sonoma, CA (US); Marja Koivunen, Davis, CA (US); Pamela Marrone, Davis, CA (US); Huazhang Huang, Woodland, CA (US); Ratnakar Asolkar, Davis, CA (US)

(73) Assignee: MARRONE BIO INNOVATIONS, INC., Davis, CA (US)

(21) Appl. No.: 13/294,918

(22) Filed: Nov. 11, 2011

Related U.S. Application Data
(60) Provisional application No. 61/413,435, filed on Nov. 13, 2010.

(51) Int. Cl. A01N 63/02; A01P 9/00; C07D 305/12; C07C 57/03; C07C 59/44

(52) U.S. Cl. 424/780; 554/223; 554/219; 549/323

(57) ABSTRACT

Compositions and methods for controlling Limnoperna sp., particularly, golden mussels are provided using Pseudomonas sp. suspension or compounds and compositions derived from said suspension.

### TECHNICAL FIELD

Compositions and methods for controlling Limnoperna sp., particularly, golden mussels are provided using Pseudomonas sp. suspension or compounds and compositions derived from said suspension.

### BACKGROUND

The ability of the mussels to quickly colonize new areas, rapidly achieve high densities and attach to any hard substratum (e.g., rocks, logs, aquatic plants, shells of native mussels, exoskeletons of crayfish, plastic, concrete, wood, fiberglass, pipes made of iron and polyvinyl chloride and surfaces covered with conventional paints) make it possible for them to cause serious adverse consequences. These consequences include damage to water-dependent infrastructure resulting in millions of dollars increase in the operating expense and significant damage to the ecological systems.

Management of mussels is very important for protecting water-dependent infrastructure and aquatic ecological systems. There are many proactive and reactive methods to control and reduce the populations of mussels. Reactive removal includes the mechanical removal, predator removal, and chemical and biochemical removal of adult mussels. For example, fish, birds, crayfish, crabs, leeches and mammals have shown to predate mussels. However, it is unlikely that invasive mussel populations will be controlled by natural predation, especially in man-made structures such as pipes or pumping plants. Proactive measures to control mussels includes any mechanical, physical or chemical means in witch the planktonic (veliger) mussel life stage is prevented from settling and growing into the adult life stage or colonizing on hard substrates. Preventing mussels from colonizing and growing into adult life stages is also referred to as settlement prevention.

The Golden Mussel, Limnoperna fortunei, is indigenous to China. It was first detected in Hong Kong in the late 1960s and has since fouled water systems in many Asian countries such as Japan, China, South Korea and Taiwan. It was introduced in Argentina in 1991 and has been detected in a number of other South American countries (e.g., Brazil, Paraguay, Columbia, Argentina, Uruguay (see Ricciardi, 1998, Biofouling 13:97-106 and Darrigan, 2003, Tentacle No. 11, pp. 8-9 for review). Research indicates that it is likely that if introduced into the southern regions of North America, that L. fortunei could infest waterways throughout the southeastern and southwestern regions of North America (Oliveira et al., 2010, Aquatic Invasions 5: 59-73). It appears to have a great impact in the biofouling throughout the fish aquaculture industry, water transfers and conveyance, in the water intakes of industrial, electric power and drinking water plants.

Application of molluscicides is one effective way to reduce the mussel population. For example, sodium hypochlorite is a commonly used control agent in Europe, US, and Canada. However, mussels can withstand this treatment for several days by closing their shells and chlorine can be only used in pipes or ducts that contain pressure-sensing or other sensory equipment due to environmental toxicity of chlorine (U.S. Army Engineer Waterways Experiment Station. 1995. Zebra mussels: Biology, Ecology, and Recommended Control Strategies. Technical Note. ZMR-1-01. Zebra Mussel Research Program, Vicksburg, Mass.). In addition, there are many other commercialized molluscicides such as surfactant ammonium salts, Butylated hydroxytoluene (BHT) in paints, N-triphenylmethyl-morpholine and so on. These chemicals have either low selectivity or affect the water ecosystems. For example, a 4-trifluroethyl-4-nitrophenol marketed as Bayluscide® (Bayer) is a possible candidate for control of such invasive exotic species. However, the mode of action of this chemical is to affect mussel cellular respiration, which in nature will have a limited selectivity between mussels and other aquatic species such as fish (Karen Perry and John Lynn, Detecting physiological and pesticide-induced apoptosis in early developmental stages of invasive bivalves, Hydrobiologia (2009) 628:153-164; I Takougang, J Meli, F Angwafo, Field trials of low dose Bayluscide on snail hosts of schistosome and selected non-target organisms in sahelian Cameroon, Mem Inst Oswaldo Cruz, Rio de Janeiro, 2006, 101(4): 355-358).

Microbes have also been used to control molluscs (see, for example, WO93/00816 and WO91/00012). An isolate, strain CL145A of Pseudomonas fluorescens, has been found to be lethal to zebra mussels (see Molloy, D. P. U.S. Pat. No. 6,194,194, issued Feb. 27, 2001). US Patent Application Pub. No. 20100266717 discloses compounds derived from Pseudomonas fluorescens, which may be used to control zebra and quagga mussels.

### BRIEF SUMMARY OF THE DISCLOSURE

Provided is a method for controlling Limnoperna species, in a location (e.g., a liquid wherein said liquid is water such as a body of water or flowing water; paint and/or a solid surface which includes but is not limited to plastic, concrete, wood, fiberglass, pipes made of iron and polyvinyl chloride, surfaces covered with coating materials and/or paints) where control is desired comprising introducing into said location an amount of a cell suspension comprising cells having the toxin-producing characteristics of a Pseudomonas species effective to control said Limnoperna species and/or an amount of one or more substances which are (a) toxic to Limnoperna species and (b) derived from said Pseudomonas species or cell suspension derived from said Pseudomonas species. The substance in a particular embodiment may be a peptide, protein lipid and/or lactone. In an even more particular embodiment, the substance may be those compounds disclosed in US Patent Application Pub. No. 20100266717, the contents of which are incorporated herein by reference and set forth below.

In another particular embodiment, the substance may be a compound that (a) has molluscidal activity; (b) has a molecular weight of about 280-320 as determined by Liquid Chromatography/Mass Spectroscopy (LC/MS) and (c) has an High Pressure Liquid Chromatography (HPLC) retention time of about 13-20 minutes on a reversed phase C-18 HPLC column using a water:acetonitrile (CH₃CN) gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0.5 mL/min flow rate and UV detection of 210 nm and the compound in one embodiment may be a hydroxyl unsaturated fatty acid.

In a particular embodiment, the compound may be derived from Pseudomonas fluorescens and characterized as having a unsaturated fatty acid structure comprising at least one carboxylic acid moiety, at least one unsaturated moiety and at least one methyl group; molecular weight from 230 to about 270 in the core structure; at least 5 carbons and at least 2 oxygens.

In yet another particular embodiment, the substance may be a compound that (a) has molluscidal activity; (b) has a molecular weight of about 280-320 as determined by Liquid Chromatography/Mass Spectroscopy (LC/MS) and (c) has an High Pressure Liquid

Chromatography (HPLC) retention time of about 13-20 minutes on a reversed phase C-18 HPLC column using a water:acetonitrile (CH₃CN) gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0.5 mL/min flow rate and UV detection of 210 nm and the compound in one embodiment may be a hydroxyl unsaturated fatty acid.

In yet another particular embodiment, the compound may be derived from Pseudomonas fluorescens and characterized as having a hydroxylated unsaturated fatty acid structure comprising at least one carboxylic acid moiety, at least one unsaturated moiety and at least one alcohol group; molecular weight from 280 to about 320 in the core structure; at least 15 carbons and at least 3 oxygens.

In a related aspect, also provided is the use of said cell suspension and/or substance(s) derived from a Pseudomonas species, particularly from said suspension to control Limnoperna species. Further provided are compositions comprising said cell suspension and/or substance(s) derived from a Pseudomonas species for controlling Limnoperna species.

### DETAILED DESCRIPTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As defined herein, “controlling Limnoperna species” means controlling the eggs, larvae, veligers and post-veligers of the golden mussel by killing or disabling them so that they cannot colonize, grow, establish, or reproduce in a given location.

As defined herein, “derived from” and “obtainable from” means directly isolated or obtained from a particular source or alternatively having identifying characteristics of a substance or organism isolated or obtained from a particular source. These terms are used interchangeably throughout the specification.

As defined herein, an “isolated compound” is essentially free of other compounds or substances, e.g., at least about 20% pure, preferably at least about 40% pure, more preferably about 60% pure, even more preferably about 80% pure, most preferably about 90% pure, and even most preferably about 95% pure, as determined by analytical methods, including but not limited to chromatographic methods, electrophoretic methods.

### Substances

The substances used in the compositions and methods of the set forth above may be derived from Pseudomonas species. The substances may be compounds such as peptides, proteins and/ or lactones.

Examples of such substances are disclosed in US Patent Application Pub. No. 20100266717, the contents of which are incorporated herein by reference and include but are not limited to:

(I) a compound that (a) has a molecular weight of about 1280-1310 as determined by Liquid Chromatography/Mass Spectroscopy (LC/MS); (b) has 1H NMR values of δ 9.25, 8.36, 8.06, 7.82, 7.71, 7.52, 7.45, 6.82, 6.36, 6.08, 5.42, 5.39, 5.30, 5.14, 4.68, 4.42, 4.31, 4.16, 4.11, 4.07, 3.95-3.86, 3.83, 3.72, 3.66, 3.53, 3.48, 3.37, 3.17, 3.06, 2.56, 2.53, 2.45, 2.32, 2.21, 2.02, 1.96, 1.84, 1.72, 1.65, 1.61, 1.51, 1.48-1.37, 1.32, 1.12, 0.94, 0.91, 0.68; (c) has an (High Pressure Liquid Chromatography) (HPLC) retention time of about 50-55 min on a reversed phase C-18 HPLC column using a water:acetonitrile gradient solvent system (0-10 min; 30-40% aqueous CH₃CN, 10-20 min; 40-60% aqueous CH₃CN, 20-60 min; 60-80% aqueous CH₃CN, 60-65 min; 80-100% aqueous CH₃CN) at 2.5 mL/min flow rate and UV detection of 210 nm;

(II) a compound that has (a) a molecular weight of about 1310-1335, more particularly, about 1321 as determined by LC/MS; (b) has an HPLC retention time of about 55-60 min on a reversed phase C-18 HPLC column using a water: acetonitrile gradient solvent system (0-10 min; 30-40% aqueous CH₃CN, 10-20 min; 40-60% aqueous CH₃CN, 20-60 min; 60-80% aqueous CH₃CN, 60-65 min; 80-100% aqueous CH₃CN) at 2.5 mL/min flow rate and UV detection of 210 nm;

(III) a substance present in a composition comprising a water: acetonitrile solvent system (0-10 min; 35-45% aqueous CH₃CN, 10-20 min; 45-60% aqueous CH₃CN, 20-50 min; 60-85% aqueous CH₃CN, 50-60 min; 85-100% aqueous CH₃CN, 60-70 min; 100% CH₃CN) at 10 mL/min flow rate and UV detection of 210 nm fraction obtainable from a Pseudomonas species cell suspension by HPLC with a retention time of about 45-50 min, said fraction comprising at least two compounds that (i) are toxic to a member of a Gastropoda and/or Bivalvia class; (ii) have molecular weights between about 630-660, preferably about 643 and between about 970-1000, preferably about 984, as determined by LC/MS;

(IV) a compound that (a) has a molecular weight of about 540-550 as determined by LC/MS; (b) has an HPLC retention time of about 50-55 min on a reversed phase C-18 HPLC column using a water: acetonitrile solvent system (0-10 min; 35-45% aqueous CH₃CN, 10-20 min; 45-60% aqueous CH₃CN, 20-50 min; 60-85% aqueous CH₃CN, 50-60 min; 85-100% aqueous CH₃CN, 60-70 min; 100% CH₃CN) at 10 mL/min flow rate and UV detection of 210 nm.

In a particular embodiment, the compound may be derived from Pseudomonas fluorescens and has a hydroxylated unsaturated fatty acid lactone structure comprising at least one lactone moiety which is a 5 membered y-lactone, at least one unsaturated moiety and at least one alcohol group; a molecular weight from 285 to about 310 in the core structure; at least 15 carbons and at least 3 oxygens. In a more particular embodiment, the compound may have the structure

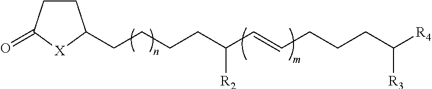

wherein: X are each independently —O, —NR₁, or —S, wherein R₁ is —H or C₁-C₆ alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond or triple bond. In yet another particular embodiment, Y and M are oxygen, A and X are carbon and n is 2 or 3, R is a C7 or C8 alkyl and z is 0, wherein when n is 2 and R is a C7 alkyl, R is attached to A.

In a particular embodiment, the compound is piliferolide A

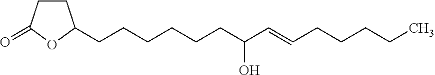

In yet another particular embodiment, the compound may be derived from Pseudomonas fluorescens and characterized as having a hydroxylated unsaturated fatty acid structure comprising at least one carboxylic acid moiety, at least one unsaturated moiety and at least one alcohol group; molecular weight from 285 to about 310 in the core structure; at least 15 carbons and at least 3 oxygens.

In a more particular embodiment, there are provided compounds having the structure

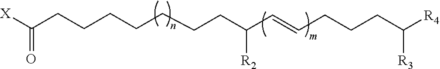

wherein: X are each independently —OH, —NR₁, or —S, wherein R₁ is —H or C₁-C₆ alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond, triple bond, with a molecular weight of between about 285 to about 310.

In a most specific embodiment, the compound is 11-hydroxy-12-ene-octadecanoic acid and has the structure

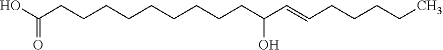

Other compounds disclosed in US Patent Application Pub. No. 20100266717 include but are not limited to:
(a) a lactone selected from the group consisting of gamma-dodecalactone, delta-tridecalactone, piliferolide A and alpha-heptyl-gamma-butyrolactone and
(b) a sarmentine analog selected from the group consisting of N-Cyclopentyldecanamide, N-(Decanoyl)pyrrolidine, N-(Decanoyl)piperidine, N-(Decanoyl)hexamethyleneimine, N-Cyclopentyldecenamide, (N-(Decenoyl)pyrrolidine, N-(Decenoyl)piperidine, N-(Decenoyl)hexamethyleneimine and N-(Decenoyl)piperidine and
(c) 11-hydroxy-12-ene-octadecanoic acid.

In addition to the compounds disclosed in US Patent Application Pub. No. 20100266717, the substance may be a compound that (a) has molluscidal activity; (b) has a molecular weight of about 230-270 as determined by Liquid Chromatography/Mass Spectroscopy (LC/MS) and (c) has an High Pressure Liquid Chromatography (HPLC) retention time of about 16-25 minutes on a reversed phase C-18 HPLC column using a water:acetonitrile (CH₃CN) gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0.5 mL/min flow rate and UV detection of 210 nm and. the compound in one embodiment may be a unsaturated fatty acid.

In a particular embodiment, the compound may be derived from Pseudomonas fluorescens and characterized as having a unsaturated fatty acid structure comprising at least one carboxylic acid moiety, at least one unsaturated moiety and at least one methyl group; molecular weight from 230 to about 270 in the core structure; at least 5 carbons and at least 2 oxygens.

In one specific embodiment, the compound (a) is obtainable from a Pseudomonas sp (b) is toxic to Limnoperna species (c) has molecular weight of about 240-265 and more particularly, 254 as determined by Liquid Chromatography/Mass Spectroscopy (LC/MS); (d) has ¹H NMR values of δ 5.35, 2.28, 2.04, 1.61, 1.34, 1.31, 0.91 and has ¹³C NMR values of δ 176.63, 129.75, 129.64, 33.86, 31.81, 29.73, 29.68, 29.20, 29.13, 29.08, 28.94, 27.05, 27.00, 24.98, 22.62, 13.35 (e) has an High Pressure Liquid Chromatography (HPLC) retention time of about 16-25 minutes, more specifically about 20 minutes and even more specifically about 20.18 min on a reversed phase C-18 HPLC (Phenomenex, Luna 5μ C18(2) 100 A, 100×4.60 mm) column using a water:acetonitrile (CH₃CN) with a gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0.5 mL/min flow rate and UV detection of 210 nm.

In a more particular embodiment, there are provided compounds including but not limited to:

(A) a compound having the structure

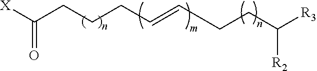

or a acceptable salt or steriosomers thereof, wherein: X are each independently —OH, —NR₁, or —S, wherein R₁ is —H or C₁-C₆ alkyl; n=0 to 15, R₂, R₃ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond, triple bond.

(B) a compound having the structure

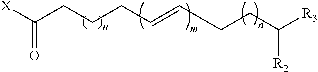

or a acceptable salt or steriosomers thereof, wherein: X are each independently —OH, —NR₁, or —S, wherein R₁ is —H or C₁-C₆ alkyl; n=0 to 15, R₂, R₃ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=0 to 15,

In a most specific embodiment, the compound is 9-hexadecenoic acid

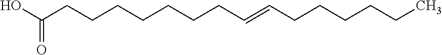

In yet another embodiment, the compound may be a compound that (a) has molluscidal activity; (b) has a molecular weight of about 280-320 as determined by Liquid Chromatography/Mass Spectroscopy (LC/MS) and (c) has an High Pressure Liquid Chromatography (HPLC) retention time of about 13-20 minutes on a reversed phase C-18 HPLC column using a water:acetonitrile (CH₃CN) gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0 5 mL/min flow rate and UV detection of 210 nm and the compound in one embodiment may be a hydroxyl unsaturated fatty acid.

In a particular embodiment, the compound may be derived from Pseudomonas fluorescens and characterized as having a hydroxylated unsaturated fatty acid structure comprising at least one carboxylic acid moiety, at least one unsaturated moiety and at least one alcohol group; molecular weight from 280 to about 320 in the core structure; at least 15 carbons and at least 3 oxygens.

In one specific embodiment, the compound (a) is obtainable from a Pseudomonas sp (b) is toxic to Limnoperna species (c) has molecular weight of about 280-320 and more particularly, 298 as determined by Liquid Chromatography/Mass Spectroscopy (LC/MS); (d) has ¹H NMR values of δ 5.57, 5.42, 3.65, 2.36, 2.23, 2.06, 1.65, 1.49, 1.33, 0.90 and has ¹³C NMR values of δ 179.41, 133.23, 125.13, 71.51, 36.73, 35.26, 33.98, 31.81, 29.48, 29.32, 29.02, 28.99, 28.91, 27.32, 25.67, 24.64, 22.62, 14.08 (e) has an High Pressure Liquid Chromatography (HPLC) retention time of about 13-20 minutes, more specifically about 16 minutes and even more specifically about 16.81 min on a reversed phase C-18 HPLC (Phenomenex, Luna 5μ C18(2) 100 A, 100×4.60 mm) column using a water:acetonitrile (CH₃CN) with a gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0.5 mL/min flow rate and UV detection of 210 nm.

In a more particular embodiment, there are provided compounds including but not limited to:

(A) a compound having the structure

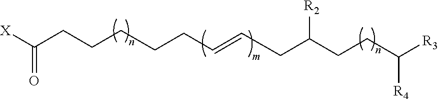

or a acceptable salt or steriosomers thereof, wherein: X are each independently —OH, —NR_{S}, or —S, wherein R₁ is —H or C₁-C₆alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond, triple bond.

(B) a compound having the structure

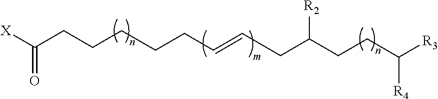

wherein R₁ is —H or C₁-C₆alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=0 to 15.

In a particular embodiment, the compound is ricinoleic acid

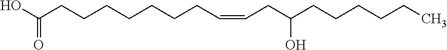

### Methods of Production

As noted above, the compounds and compositions may be obtained, is obtainable or derived from an organism having the identifying characteristics of a Pseudomonas species, more particularly, from an organism having the identifying characteristics of a strain of Pseudomonas fluorescens or alternatively from an organism having the identifying characteristics of Pseudomonas fluorescens isolate, ATCC 55799 as set forth in U.S. Pat. No. 6,194,194. The methods comprise cultivating these organisms and optionally obtaining the compounds by isolating these compounds from the cells of these organisms.

In particular, the organisms are cultivated in a nutrient medium using methods known in the art. The organisms may be cultivated by shake flask cultivation, small scale or large-scale fermentation (including but not limited to continuous, batch, fed-batch, or solid state fermentations) in laboratory or industrial fermentors performed in suitable medium and under conditions allowing cell growth. The cultivation may take place in suitable nutrient medium comprising carbon and nitrogen sources and inorganic salts, using procedures known in the art. Suitable media are available may be available from commercial sources or prepared according to published compositions. A particular embodiment is disclosed in the examples infra and in U.S. Pat. No. 6,194,194.

After cultivation, the cells may be concentrated and subsequently suspended in a buffer to obtain a cell suspension. In one embodiment, a suspension of dead cells is used. Live cells in the cellular suspension may be killed by at least one of the following: irradiating, heating, drying, r treating cells with other chemical of physical means. A dead cell suspension is not required for activity against mussel species.

In a particular embodiment, substances toxic to Limnoperna species may be extracted from the suspension. The extract may be fractionated by chromatography. Chromatographic fractions may be assayed for toxic activity against molluscs, such as mussels, snails (e.g., aquatic and/or garden snails) and/or slugs, using methods known in the art; one particular embodiment is disclosed in the examples, infra. This process may be repeated one or more times using the same or different chromatographic methods.

### Compositions

The cell suspensions and/or compounds may be formulated into a composition. The compositions may be in the form of a powder, suspension, a solution, an emulsion, a dusting powder, a dispersible granule, a wettable powder, an emulsifiable concentrate, an aerosol or impregnated granule, formulated by techniques well known to those skilled in the art. In any formulation described herein, percent of active ingredient is within a range of 0.01% to 99.99%.

Additives to these compositions may include but are not limited to surface active agents, inert carriers, preservatives, humectants, feeding stimulants, attractants, encapsulating agents, binders, emulsifiers, dyes, UV protectants, buffers, flow agents, or other components which facilitate product handling and application.

The compositions of this invention may also be formulated as active mixtures which may include finely divided dry or liquid diluents, extenders, fillers, conditioners, and excipients, including various clays, diatomaceous earth, talc and the like, or water and various organic liquids and mixtures thereof.

Inert ingredients may be added to stabilize and disperse the product in, for example, water. Examples of the inert material that may be used in the compositions of the present invention include, but are not limited to, inorganic minerals such as kaolin, mica, gypsum, phyllosilicates, carbonates, sulfates, or phosphates; or botanical materials such as wood products, cork, powdered corn cobs, rice hulls, peanut hulls and walnut shells. In a particular embodiment, the inert material can be obtained or derived from a clay mineral (kaolinite, smectite, attapulgite) suspended in water at a rate of about 1 to 20 mg/liter corresponding to approximately 1 to 20 NTU (normalized turbidity units). The inert materials used to enhance mussel siphoning can be applied in solid form or as a suspension in aqueous solution, preferably water, directly to the water or the location (e.g., solid surface) where the mussels are treated. In a particular embodiment, to enhance product efficacy, an inert material such as clay, silt, sediment or any other material with no nutritional value and with a small enough particle size can be suspended in water prior to the treatment with the active ingredient.

### Methods of Use

The cell suspensions, compounds and compositions set forth above may be used to control Limnoperna species, in particular, Limnoperna fortunei (golden mussel) in water, such as a body of water or flowing water, including but not limited to, water flows confined in enclosed or semi-enclosed conveyance structures or on surfaces where Limnoperna species gather or alternatively as an anti-fouling agent in paint. In the event that it is used as an antifouling agent in paint, it is present in an anti-vegetative, biocidally effective amount. Surfaces where Limnoperna species gather include but are not limited to plastic, concrete, wood, fiberglass, pipes made of iron and polyvinyl chloride and surfaces covered with paints and/or coatings. Coatings may be formulated from pigments, binders, additives, and/or carrier fluids and are preferably applied in a thin film to provide protection or decoration to a surface. The end product (which contains the active compound) will be used at 10-500 mg/L, more specifically at 25-500 mg/L (ppm) or 25-50000 mg/kg. It will be applied either as a dry product or suspended in water into pipes, conveyance structures, water intake structures, dam structures, holding tanks, and open waters such as streams, rivers, lakes, irrigation canals, ponds and lakes through specific application pumps and mixing systems.

### EXAMPLES

The Examples below is presented to describe preferred embodiments and utilities of the invention and is not meant to limit the invention unless otherwise stated in the claims appended hereto.

### Example 1

### Effect of Pseudomonas Fluorescens, ATCC 55799 Suspension of Killed Cells on Golden Mussels, Limnoperna Fortunei

### Mussel Collection:

Day 1, Mussel Collection: mussels were collected from the Paraguay River and put in aquarium with aeration and filtration system, pH=7.0; T=25.5° C.; turbidity=14NTU.

### Experimental Setup:

Day 1, Acclimation: 20 mussels in each jar with 250 ml of natural water from the Paraguay River. Four jars for control, and 5 jars for treatment. Jars were kept in the incubator about 25-26° C. with aeration.

Day 2, Treatment: Water was changed and product was dosed at a product dilution of 2.5 g to 500 ml of deionized water for a concentration of 5 g/L as DCW (dry cell weight) Jar Assay Conditions: 10 mL to 250 mL jar with river water to achieve a dose of 200 mg/L as DCW. Mussels were held in treated water for 24 hours and compared to untreated control for 24 hours. Jars were constantly aerated for the entire 24 hour period.

Conditions in test jars containing water and Pseudomonas fluorescens, ATCC 55799 suspension of killed cells: T=26° C., pH=7.0, Turbidity=57.0 NTU Observation: mussels were filtering after the product was dosed.

Day 3, Post Treatment: Mussels in both treated and control jars were taken from the jars, washed and transferred to Petri dish with fresh water.

Conditions in test jars containing water and Pseudomonas fluorescens, ATCC 55799 suspension of killed cells day after the treatment: T=26° C., pH=7.12, Turbidty=17.5 NTU

### Mortality Results:

T 1-5: Treatment and C 1-4: no addition of Pseudomonas fluorescens, ATCC 55799 suspension of killed cells

Results are shown in Table I below:

TABLE I

| Day | T1 | T2 | T3 | T4 | T5 | C1 | C2 | C3 | C4 |
|---|---|---|---|---|---|---|---|---|---|
| 1 | 0 | 1 | 2 | 0 | 0 | 0 | 0 | 0 | 0 |
| 2 | 2 | 2 | 2 | 0 | 2 | 0 | 0 | 0 | 0 |
| 3 | 1 | 4 | 3 | 1 | 0 | 0 | 0 | 0 | 0 |
| 4 | 1 | 3 | 0 | 0 | 2 | 0 | 0 | 0 | 0 |
| 5 | 0 | 1 | 0 | 0 | 0 | 0 | 0 | 1 | 0 |
| 6 | 0 | 0 | 1 | 0 | 0 | 0 | 0 | 0 | 0 |
| 7 | 0 | 0 | 0 | 0 | 0 | 0 | 0 | 0 | 0 |
| 8 | 0 | 0 | 0 | 0 | 0 | 0 | 0 | 0 | 0 |
| 9 | 0 | 0 | 0 | 0 | 0 | 0 | 0 | 0 | 0 |
| 10 | 0 | 0 | 0 | 0 | 0 | 0 | 0 | 0 | 0 |
| 11 | 0 | 0 | 0 | 0 | 0 | 0 | 0 | 0 | 0 |
| total | 4 | 11 | 8 | 1 | 4 | 0 | 0 | 1 | 0 |
| Percent | 20 | 55 | 40 | 5 | 20 | 0 | 0 | 5 | 0 |
| Mortality | | | | | | | | | |

Average mussel mortality in treated jars: 28%; Average control mortality: 0.25%.

Behavioral differences were observed between mussels in treated water and mussels in the control. Treated mussels did not completely close their shells. Even with outside stimulation the shells remained open and were slow to close after being touched. Mussels in the control jars remained tightly closed and opened just enough to put the siphon out. Control mussels quickly reacted to probing and outside stimulation by closing their shells.

### Example 2

### Extraction of the Compounds from Pseudomonas Fluorescens

The following procedure is used for the purification of compounds extracted from the culture of Pseudomonas fluorescens:

The culture broth derived from the 10-L fermentation P. fluorescens in FM2-growth media is first centrifuge to separate the cells pellet and supernatant. The supernatant and the cell pellet were extracted separately using Amberlite XAD-7 resin (Asolkar et al., 2006) by shaking the cell suspension/supernatant with resin at 225 rpm for two hours at room temperature. The resin and cell mass are collected by filtration through cheesecloth and washed with DI water to remove salts. The resin, cell mass, and cheesecloth are then soaked for 2 h in acetone/methanol (50/50) after which the acetone/methanol is filtered and dried under vacuum using rotary evaporator to give two separate crude extract.(one for supernatant and other for cell pellets). The crude extracts were then fractionated separately by using reversed-phase C18 vacuum liquid chromatography (H₂O/CH₃OH; gradient 90:20 to 0:100%) to give 7 fractions. These fractions are then concentrated to dryness using rotary evaporator and the resulting dry residues are screened for biological activity using both a live mussel jar-test bioassay with quagga mussels as well as a cell-based assay with a freshwater snail embryo cell line (Biomphalaria glabrata). The active fractions are then subjected to reversed phase HPLC (Spectra System P4000 (Thermo Scientific) to give pure compounds, which are then screened in above mentioned bioassays to locate/identify the active compounds. To confirm the identity of the compound, additional spectroscopic data such as LC/MS and NMR is recorded.

9-hexadecenoic acid was obtained from fraction 4 obtained from the fractionation of cell pellet crude extract whereas ricinoleic acid was obtained from fraction 3 that was obtained from the fractionation of crude extract obtained from the extraction of supernatant.

### Purification of Compounds

Purification of 9-hexadecenoic acid was performed by using HPLC C-18 column (Phenomenex, Luna 10u C18(2) 100 A, 250×30), water:acetonitrile gradient solvent system (0-10 min; 50-60% aqueous CH₃CN, 10-20 min; 60-75% aqueous CH₃CN, 20-45 min; 75-100% aqueous CH₃CN, 45-55 min; 100% CH₃CN, 55-70 min; 100-50% aqueous CH₃CN) at 8 mL/min flow rate and UV detection of 210 nm. The active compound 9-hexadecenoic acid has retention time 66.17 min.

Purification of Ricinoleic acid was performed by using HPLC C-18 column (Phenomenex, Luna 10u C18(2) 100 A, 250×30), water:acetonitrile gradient solvent system (0-10 min; 50-60% aqueous CH₃CN, 10-20 min; 60-75% aqueous CH₃CN, 20-45 min; 75-100% aqueous CH₃CN, 45-55 min; 100% CH₃CN, 55-70 min; 100-50% aqueous CH₃CN) at 8 mL/min flow rate and UV detection of 210 nm. Ricinoleic acid was eluted at 48.52 min.

### Mass Spectroscopy Analysis of Compounds:

Mass spectroscopy analysis of active compounds were performed on a Thermo Finnigan LCQ Deca XP Plus electrospray (ESI) instrument using both positive and negative ionization modes in a full scan mode (m/z 100-1500 Da) on a LCQ DECA XP^{plus} Mass Spectrometer (Thermo Electron Corp., San Jose, Calif.). Thermo high performance liquid chromatography (HPLC) instrument equipped with Finnigan Surveyor PDA plus detector, autosampler plus, MS pump and a 4.6 mm×100 mm Luna C18 5 μm column (Phenomenex). The solvent system consisted of water (solvent A) and acetonitrile (solvent B). The mobile phase begins at 10% solvent B and is linearly increased to 100% solvent B over 20 min and then kept for 4 min, and finally returned to 10% solvent B over 3 min and kept for 3 min The flow rate is 0.5 mL/min. The injection volume was 10 μL and the samples are kept at room temperature in an auto sampler. The compounds are analyzed by LC-MS utilizing the LC and reversed phase chromatography. Mass spectroscopy analysis of the present compounds is performed under the following conditions: The flow rate of the nitrogen gas was fixed at 30 and 15 arb for the sheath and aux/sweep gas flow rate, respectively. Electrospray ionization was performed with a spray voltage set at 5000 V and a capillary voltage at 35.0 V. The capillary temperature was set at 400° C. The data was analyzed on Xcalibur software. The active compound 9-hexadecenoic acid has a molecular mass of 253.84 in negative ionization mode suggesting the molecular weight of 254. The LC-MS chromatogram for another compound Ricinoleic acid suggests a molecular mass of 297.50 in negative ionization mode confirming the molecular weight of 298.

### NMR Spectroscopy Analysis of Compounds

NMR-NMR spectra were measured on a Bruker 600 MHz gradient field spectrometer. The reference is set on the internal standard tetramethylsilane (TMS, 0.00 ppm). The amino acid analyses were carried out on Hitachi 8800 amino acid analyzer.

For structure elucidation, the purified 9-hexadecenoic acid with molecular weight 254 is further analyzed using a 600 MHz NMR instrument, and has ¹H NMR δ values at 5.35, 2.28, 2.04, 1.61, 1.34, 1.31, 0.91and has ¹³C NMR values of 176.63, 129.75, 129.64, 33.86, 31.81, 29.73, 29.68, 29.20, 29.13, 29.08, 28.94, 27.05, 27.00, 24.98, 22.62, 13.35 The detail 1D and 2D NMR analysis confirm the structure for the compound as 9-hexadecenoic acid as a known compound (see FIG. 9) also reported from Pseudomonas sp. strain E-3 (Okuyama H. et al., 1996, Identification of activities that catalyze the cis-trans isomerization of the double bond of a mono-unsaturated fatty acid in Pseudomonas sp. strain E-3. Arch. Microbio, 165, 415-417) and molecular formula C₁₆H₃₀O₂.

The compound purified from F3 of the supernatant crude extract with molecular weight 298 is further analyzed using a 600 MHz NMR instrument, and has ¹H NMR δ values at 5.57, 5.42, 3.65, 2.36, 2.23, 2.06, 1.65, 1.49, 1.33, 0.90 and ¹³C NMR values of δ 179.41, 133.23, 125.13, 71.51, 36.73, 35.26, 33.98, 31.81, 29.48, 29.32, 29.02, 28.99, 28.91, 27.32, 25.67, 24.64, 22.62, 14.08. The detailed 1D and 2D NMR analysis confirm the structure as ricinoleic acid (Dembitsky V. et al., 1993; Unusual hydroxyl fatty acids from some higher fungi, 34, 1057-1059) a known fatty acid to the compound with the molecular formula C₁₈H₃₄O₃.

The potency of compounds isolated from Pseudomonas broth is tested using procedures described in the US Patent Application Pub. No. 20100266717. The results are shown below in Table 2.

TABLE 2
Molluscicidal Effects of 9-hexadecenoic acid and Ricinoleic acid

| | | % | % | % | % |
|---|---|---|---|---|---|
| | Conc | mortality | mortality | mortality | mortality |
| structure | [μg/ml] | 24 hr | 48 hr | 72 hr | 96 hr |
| 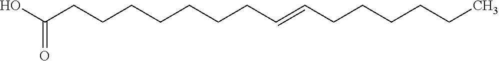 | 16 | 20 ± 14.1 | 25.5 ± 7.1 | 30 ± 14.1 | 30 ± 14.1 |
| 9-hexadecenoic acid | | | | | |
| 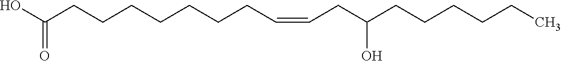 | 16 | 20 ± 0 | 20 ± 0 | 20 ± 0 | 20 ± 0 |
| Ricinoleic acid | | | | | |

Although this invention has been described with reference to specific embodiments, the details thereof are not to be construed as limiting, as it is obvious that one can use various equivalents, changes and modifications and still be within the scope of the present invention.

Various references are cited throughout this specification, each of which is incorporated herein by reference in its entirety.

What is claimed is:

1. A method for controlling Limnoperna species, in a location where control is desired comprising introducing into said location an amount of at least one of (a) a cell suspension comprising cells having the toxin-producing characteristics of a Pseudomonas species; (b) one or more substances which are (a) toxic to Limnoperna species and (b) is derived from a Pseudomonas species or cell suspension derived from a Pseudomonas species effective to control said Limnoperna species.

2. The method according to claim 1, wherein the cell suspension comprises cells of Pseudomonas fluorescens or mutant strain thereof.

3. The method according to claim 1, wherein said cell suspension comprises dead bacterial cells.

4. The method according to claim 1, wherein said substances are derived from a Pseudomonas species strain, having the identifying characteristics of P. fluorescens ATCC 55799.

5. The method according to claim 1, wherein said substance is a protein, peptide, lipid or lactone.

6. The method according to claim 1, wherein said substance is selected from the group consisting of:
(a) a compound that (i) has a molecular weight of about 1280-1310 as determined by Liquid Chromatography/Mass Spectroscopy (LC/MS); (ii) has 1H NMR values of δ 9.25, 8.36, 8.06, 7.82, 7.71, 7.52, 7.45, 6.82, 6.36, 6.08, 5.42, 5.39, 5.30, 5.14, 4.68, 4.42, 4.31, 4.16, 4.11, 4.07, 3.95-3.86, 3.83, 3.72, 3.66, 3.53, 3.48, 3.37, 3.17, 3.06, 2.56, 2.53, 2.45, 2.32, 2.21, 2.02, 1.96, 1.84, 1.72, 1.65, 1.61, 1.51, 1.48-1.37, 1.32, 1.12, 0.94, 0.91, 0.68 and (c) has an (High Pressure Liquid Chromatography) (HPLC) retention time of about 50-55 min on a reversed phase C-18 HPLC column using a water:acetonitrile gradient solvent system (0-10 min; 30-40% aqueous CH₃CN, 10-20 min; 40-60% aqueous CH₃CN, 20-60 min; 60-80% aqueous CH₃CN, 60-65 min; 80-100% aqueous CH₃CN) at 2.5 mL/min flow rate and UV detection of 210 nm;
(b) a compound that (i) has a molecular weight of about 1310-1335 as determined by LC/MS; (ii) has an HPLC retention time of about 55-60 min on a reversed phase C-18 HPLC column using a water: acetonitrile gradient solvent system (0-10 min; 30-40% aqueous CH₃CN, 10-20 min; 40-60% aqueous CH₃CN, 20-60 min; 60-80% aqueous CH₃CN, 60-65 min; 80-100% aqueous CH₃CN) at 2.5 mL/min flow rate and UV detection of 210 nm;
(c) a compound that (i) has a molecular weight of about 540-550 as determined by LC/MS; (ii) has an HPLC retention time of about 50-55 min on a reversed phase C-18 HPLC column using a water: acetonitrile solvent system (0-10 min; 35-45% aqueous CH₃CN, 10-20 min; 45-60% aqueous CH₃CN, 20-50 min; 60-85% aqueous CH₃CN, 50-60 min; 85-100% aqueous CH₃CN, 60-70 min; 100% CH₃CN) at 10 mL/min flow rate and UV detection of 210 nm;
(d) a composition comprising a water: acetonitrile solvent system (0-10 min; 35-45% aqueous CH₃CN, 10-20 min; 45-60% aqueous CH₃CN, 20-50 min; 60-85% aqueous CH₃CN, 50-60 min; 85-100% aqueous CH₃CN, 60-70 min; 100% CH₃CN) at 10 mL/min flow rate and UV detection of 210 nm fraction obtainable from a Pseudomonas species cell suspension by HPLC with a retention time of about 45-50 min, said fraction comprising at least two compounds that (i) are toxic to a member of a Gastropoda and/or Bivalvia class; (ii) have molecular weights between about 630-660 and between about 970-1000 as determined by LC/MS;
(e) compound that is a lactone and has a hydroxylated unsaturated fatty acid lactone structure comprising at least one lactone moiety which is a 5 membered γ-lactone, at least one unsaturated moiety and at least one alcohol group; a molecular weight from 285 to about 310 in the core structure; at least 15 carbons and at least 3 oxygens;
(f) a compound that has the structure 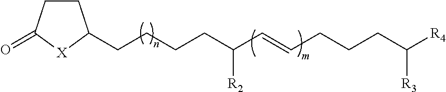
wherein: X are each independently —O, —NR₁, or —S, wherein R₁ is —H or C₁-C₆alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond or triple bond;
(g) a compound that has a hydroxylated unsaturated fatty acid structure comprising at least one carboxylic acid moiety, at least one unsaturated moiety and at least one alcohol group; a molecular weight from 285 to about 310 in the core structure; at least 15 carbons and at least 3 oxygens;
(h) a compound has the structure 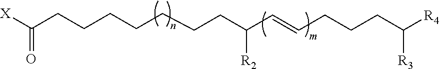
wherein: X are each independently —OH, —NR₁, or —S, wherein R, is —H or C₁-C₆ alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond, triple bond;
(i) a compound that (i) is obtainable from a Pseudomonas sp; (ii) is toxic to Limnoperna species (iii) has molecular weight of about 240-265 as determined by Liquid Chromatography/Mass Spectroscopy (LC/MS); (iv) has ¹H NMR values of δ 5.35, 2.28, 2.04, 1.61, 1.34, 1.31, 0.91 and has ¹³C NMR values of δ 176.63, 129.75, 129.64, 33.86, 31.81, 29.73, 29.68, 29.20, 29.13, 29.08, 28.94, 27.05, 27.00, 24.98, 22.62, 13.35 (v) has an High Pressure Liquid Chromatography (HPLC) retention time of about 16-25 minutes, on a reversed phase C-18 HPLC (Phenomenex, Luna 5μ C18(2) 100 A, 100×4.60 mm) column using a water:acetonitrile (CH₃CN) with a gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0 5 mL/min flow rate and UV detection of 210 nm;
(j) a compound having the structure 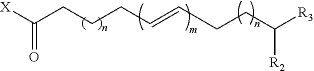
or a acceptable salt or steriosomers thereof, wherein: X are each independently —OH, —NR₁, or —S, wherein R₁ is —H or C₁-C₆ alkyl; n=0 to 15, R₂, R₃ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond, triple bond;
(k) a compound having the structure 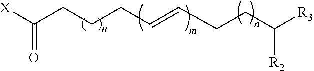
or a acceptable salt or steriosomers thereof, wherein: X are each independently —OH, —NR₁, or —S, wherein R₁ is —H or C₁-C₆ alkyl; n=0 to 15, R₂, R₃ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=0 to 15,
(l) a compound (i) is obtainable from a Pseudomonas sp (ii) is toxic to Limnoperna species (iii) has molecular weight of about 280-320 and more particularly, 298 as determined by Liquid Chromatography/Mass Spectroscopy (LC/MS); (iv) has ¹H NMR values of δ 5.57, 5.42, 3.65, 2.36, 2.23, 2.06, 1.65, 1.49, 1.33, 0.90 and has ¹³C NMR values of 67 179.41, 133.23, 125.13, 71.51, 36.73, 35.26, 33.98, 31.81, 29.48, 29.32, 29.02, 28.99, 28.91, 27.32, 25.67, 24.64, 22.62, 14.08 (v) has an High Pressure Liquid Chromatography (HPLC) retention time of about 13-20 minutes, more specifically about 16 minutes and even more specifically about 16.81 min on a reversed phase C-18 HPLC (Phenomenex, Luna 5μ C18(2) 100 A, 100×4.60 mm) column using a water:acetonitrile (CH₃CN) with a gradient solvent system (0-20 min; 90-0% aqueous CH₃CN, 20-24 min; 100% CH₃CN, 24-27 min; 0-90% aqueous CH₃CN, 27-30 min; 90% aqueous CH₃CN) at 0.5 mL/min flow rate and UV detection of 210 nm.
(m) a compound having the structure 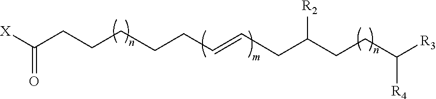
or a acceptable salt or steriosomers thereof, wherein: X are each independently —OH, —NR₁, or —S, wherein R₁ is —H or C₁-C₆ alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=double bond, triple bond;
(n) a compound having the structure 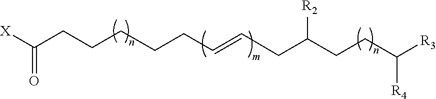
wherein R₁ is —H or C₁-C₆ alkyl; n=0 to 15, R₂ to R₄ are each independently —H, alkyl, substituted alkyl, alkenyl, substituted alkenyl, alkynyl, substituted alkynyl, aryl, substituted aryl, heteroaryl, substituted heteroaryl, heterocyclic, substituted heterocyclic, cycloalkyl, substituted cycloalkyl, alkoxy, substituted alkoxy, thioalkyl, substituted thioalkyl, hydroxy, halogen, amino, amido, carboxyl, —C(O)H, acyl, oxyacyl, carbamate, sulfonyl, sulfonamide, or sulfuryl; m=0 to 15;
(o) a lactone selected from the group consisting of gamma-dodecalactone, delta-tridecalactone, piliferolide A and alpha-heptyl-gamma-butyrolactone and
(p) a sarmentine analog selected from the group consisting of N-Cyclopentyldecanamide, N-(Decanoyl)pyrrolidine, N-(Decanoyl)piperidine, N-(Decanoyl)hexamethyleneimine, N-Cyclopentyldecenamide, (N-(Decenoyl)pyrrolidine, N-(Decenoyl)piperidine, N-(Decenoyl)hexamethyleneimine and N-(Decenoyl)piperidine;
(q) 11-hydroxy-12-ene-octadecanoic acid;
(r) 9-hexadecenoic acid and
(s) ricinoleic acid.

7. The method according to claim 1, wherein inducing death or preventing colonization controls said Limnoperna species species.

8. The method according to claim 1, wherein the Limnoperna species is L. fortunei.

9. The method according to claim 1, wherein said location is a liquid wherein said liquid is water or paint.

10. The method according to claim 1, wherein said location is water, wherein said water is a body of water or flowing water.

11. The method according to claim 1, wherein said location is a solid surface selected from the group consisting of plastic, concrete, wood, fiberglass, pipes made of iron and polyvinyl chloride, surfaces covered with coating materials and/or paints.

12. A molluscicidal composition comprising (a) a cell suspension comprising cells having the toxin-producing characteristics of a Pseudomonas species and/or (b) a substance which is (i) toxic to Limnoperna species and (ii) is derived from a Pseudomonas species or suspension derived from a Pseudomonas species to control Limnoperna species.
